(19) United States
(12) Patent Application Publication
(10) Pub. No.: US 2014/0275877 A1
(43) Pub. Date: Sep. 18, 2014

(54) SYSTEMS AND METHODS FOR DETERMINING RESPIRATION INFORMATION BASED ON PRINCIPAL COMPONENT ANALYSIS

(71) Applicants: Jimmy Dripps, West Linton (GB); Scott McGonigle, East Lothian (GB); James Ochs, Seattle, WA (US); Paul Stanley Addison, Edinburgh (GB); James Watson, Dunfermline (GB)

(72) Inventors: Jimmy Dripps, West Linton (GB); Scott McGonigle, East Lothian (GB); James Ochs, Seattle, WA (US); Paul Stanley Addison, Edinburgh (GB); James Watson, Dunfermline (GB)

(21) Appl. No.: 13/842,554

(22) Filed: Mar. 15, 2013

(51) Int. Cl. A61B 5/00; A61B 5/1455

(52) U.S. Cl. CPC A61B 5/7275; A61B 5/14551
USPC 600/323

(57) ABSTRACT

A patient monitoring system may receive a physiological signal such as a photoplethysmograph (PPG) signal. A plurality of respiration morphology signals may be determined from the PPG signal. Principal component analysis may be performed on the respiration morphology signals, resulting in one or more principal components. Respiration information such as respiration rate may be determined at least in part from a principal component that corresponds to a respiration source signal.

The present disclosure relates to physiological signal processing, and more particularly relates to determining respiration information from a physiological signal based on principal component analysis.

### SUMMARY

A method comprises receiving a photoplethysmograph (PPG) signal, processing, with processing equipment, the PPG signal to generate a plurality of respiration morphology signals, performing, with the processing equipment, principal component analysis on the plurality of respiration morphology signals to generate one or more principal components, identifying, with the processing equipment, a principal component of the one or more principal components that corresponds to a respiration source signal, and determining, with the processing equipment, respiration information based at least in part on the identified principal component.

A non-transitory computer-readable storage medium for use in determining respiration information for a patient has instructions recorded thereon for receiving a PPG signal, processing the PPG signal to generate a plurality of respiration morphology signals, performing principal component analysis on the plurality of respiration morphology signals to generate one or more principal components, identifying a principal component of the one or more principal components that corresponds to a respiration source signal, and determining respiration information based at least in part on the identified principal component.

A patient monitoring system comprises processing equipment configured to receive a PPG signal, process the PPG signal to generate a plurality of respiration morphology signals, perform principal component analysis on the plurality of respiration morphology signals to generate one or more principal components, identify a principal component of the one or more principal components that corresponds to a respiration source signal, and determine respiration information based at least in part on the identified principal component.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with some embodiments of the present disclosure;

FIG. 2 is a block diagram of the illustrative patient monitoring system of FIG. 1 coupled to a patient in accordance with some embodiments of the present disclosure;

FIG. 3 shows an illustrative PPG signal that is modulated by respiration in accordance with some embodiments of the present disclosure;

FIG. 4 shows a comparison of portions of the illustrative respiration modulated PPG signal of FIG. 3 in accordance with some embodiments of the present disclosure;

FIG. 5 shows illustrative steps for determining respiration information from a PPG signal using principal component analysis in accordance with some embodiments of the present disclosure;

FIG. 6 shows an illustrative PPG signal, a first derivative of the PPG signal, and a second derivative of the PPG signal in accordance with some embodiments of the present disclosure;

FIG. 7 shows illustrative respiration morphology signals in accordance with some embodiments of the present disclosure;

FIG. 8 shows an illustrative attractor generated from respiration morphology signals in accordance with some embodiments of the present disclosure; and

FIG. 9 shows an illustrative principal component in accordance with some embodiments of the present disclosure.

### DETAILED DESCRIPTION OF THE FIGURES

A physiological signal such as a photoplethysmograph (PPG) signal may be indicative of pulsatile blood flow. Pulsatile blood flow may be dependent on a number of physiological functions such as cardiovascular function and respiration. The PPG signal may also include modulations based on non-physiological functions such as measurement noise and patient motion.

A typical range for a patient's respiration rate (e.g., 12-40 breaths per minute) may be may be less than a typical range for a patient's pulse rate (e.g., 60-150 beats per minute). Changes to the pulsatile blood flow caused by respiration may be identified as long-term modulations to the frequency, amplitude, and baseline of the PPG signal. It may be possible to identify these long-term modulations based on patterns in the morphology of the PPG signal. A number of morphology metrics have been identified that assist in identifying these long-term modulations due to respiration. Respiration morphology signals may be generated by calculating a series of these morphology metrics over time.

Although the respiration morphology signals may better capture the respiration information, these signals may still include information from a number of sources in addition to respiration. Each of the respiration morphology signals may be thought of as a mixed signal including a respiration source signal, a pulsatile source signal, a noise source signal, and source signals due to other physiological or measurement phenomena. It may be desirable to more accurately identify the respiration source signal to better determine respiration information. As is described herein, principal component analysis (“PCA”) may be used to process a plurality of respiration morphology signals to generate one or more principal components. The principal component that corresponds to the respiration source signal may be used to determine respiration information such as respiration rate.

For purposes of clarity, the present disclosure is written in the context of the physiological signal being a photoplethysmograph signal generated by a pulse oximetry system. It will be understood that any other suitable physiological signal or any other suitable system may be used in accordance with the teachings of the present disclosure.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, β=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log Iₒ−(sβₒ+(1−s)βᵣ)1. (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( {β}_{o} ( {λ}_{IR} ) - {β}_{r} ( {λ}_{IR} ) ) - \frac{d log I ( {λ}_{IR} )}{d t} ( {β}_{o} ( {λ}_{R} ) - {β}_{r} ( {λ}_{R} ) )} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{\frac{d I}{d t}}{I} , \qquad ( 10 )$$

Eq. 8 becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} = R , \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}), (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to some embodiments, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood oxygen saturation (e.g., SpO₂), and respiration information) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may allow communication between monitor 14 and sensor unit 12.

As is described herein, monitor 14 may generate a PPG signal based on the signal received from sensor unit 12. The PPG signal may consist of data points that represent a pulsatile waveform. The pulsatile waveform may be modulated based on the respiration of a patient. Respiratory modulations may include baseline modulations, amplitude modulations, frequency modulations, baseline modulations, respiratory sinus arrhythmia, any other suitable modulations, or any combination thereof. Respiratory modulations may exhibit different phases, amplitudes, or both, within a PPG signal and may contribute to complex behavior (e.g., changes) of the PPG signal. For example, the amplitude of the pulsatile waveform may be modulated based on respiration (amplitude modulation), the frequency of the pulsatile waveform may be modulated based on respiration (frequency modulation), and a signal baseline for the pulsatile waveform may be modulated based on respiration (baseline modulation). Monitor 14 may analyze the PPG signal (e.g., by generating respiration morphology signals from the PPG signal and performing principal component analysis) to determine respiration information based on one or more of these modulations of the PPG signal.

As is described herein, respiration information may be determined from the PPG signal by monitor 14. However, it will be understood that the PPG signal could be transmitted to any suitable device for the determination of respiration information, such as a local computer, a remote computer, a nurse station, mobile devices, tablet computers, or any other device capable of sending and receiving data and performing processing operations. Information may be transmitted from monitor 14 in any suitable manner, including wireless (e.g., WiFi, Bluetooth, etc.), wired (e.g., USB, Ethernet, etc.), or application-specific connections. The receiving device may determine respiration information as described herein.

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor may emit only a Red light while a second sensor may emit only an IR light. In a further example, the wavelengths of light used may be selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which measurements may be determined based at least in part on the signal or signals received at sensor unit 12. For example, some pulse oximetry sensors rely on equations to relate an area under a portion of a PPG signal corresponding to a physiological pulse to determine blood pressure. These equations may contain coefficients that depend upon a patient's physiological characteristics as stored in encoder 42.

Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics and treatment information. In some embodiments, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; physiological characteristics (e.g., gender, age, weight); or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, data output 84, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment.”

In some embodiments, microprocessor 48 may determine the patient's physiological parameters, such as SpO₂, pulse rate, and/or respiration information, using various algorithms and/or look-up tables based on the value of the received signals and/or data corresponding to the light received by detector 18. As is described herein, microprocessor 48 may generate respiration morphology signals and perform principal component analysis on the respiration morphology signals to determine respiration information from a PPG signal.

Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable microprocessor 48 to determine the thresholds based at least in part on algorithms or look-up tables stored in ROM 52. In some embodiments, user inputs 56 may be used to enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

Data output 84 may provide for communications with other devices utilizing any suitable transmission medium, including wireless (e.g., WiFi, Bluetooth, etc.), wired (e.g., USB, Ethernet, etc.), or application-specific connections. Data output 84 may receive messages to be transmitted from microprocessor 48 via bus 50. Exemplary messages to be sent in an embodiment described herein may include samples of the PPG signal to be transmitted to an external device for determining respiration information.

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

FIG. 3 shows an illustrative PPG signal 302 that is modulated by respiration in accordance with some embodiments of the present disclosure. PPG signal 302 may be a periodic signal that is indicative of changes in pulsatile blood flow. Each cycle of PPG signal 302 may generally correspond to a pulse, such that a heart rate may be determined based on PPG signal 302. Each respiratory cycle 304 may correspond to a breath. The period of a respiratory cycle may typically be longer than the period of a pulsatile cycle, such that any changes in the pulsatile blood flow due to respiration occur over a number of pulsatile cycles. The volume of the pulsatile blood flow may also vary in a periodic manner based on respiration, resulting in modulations to the pulsatile blood flow such as amplitude modulation, frequency modulation, and baseline modulation. This modulation of PPG signal 302 due to respiration may result in changes to the morphology of PPG signal 302.

FIG. 4 shows a comparison of portions of the illustrative PPG signal 302 of FIG. 3 in accordance with some embodiments of the present disclosure. The signal portions compared in FIG. 4 may demonstrate differing morphology due to respiration modulation based on the relative location of the signal portions within a respiratory cycle 304. For example, a first pulse associated with the respiratory cycle may have a relatively low amplitude (indicative of amplitude and baseline modulation) as well as an obvious distinct dichrotic notch as indicated by point A. A second pulse may have a relatively high amplitude (indicative of amplitude and baseline modulation) as well as a dichrotic notch that has been washed out as depicted by point B. Frequency modulation may be evidence based on the relative period of the first pulse and second pulse. Referring again to FIG. 3, by the end of the respiratory cycle 304 the pulse features may again be similar to the morphology of A. Although the impact of respiration modulation on the morphology of a particular PPG signal 302 has been described herein, it will be understood that respiration may have varied effects on the morphology of a PPG signal other than those depicted in FIGS. 3 and 4.

FIG. 5 shows illustrative steps for determining respiration information from a PPG signal using principal component analysis in accordance with some embodiments of the present disclosure. Although exemplary steps are described herein, it will be understood that steps may be omitted and that any suitable additional steps may be added for determining respiration information using principal component analysis. Although the steps described herein may be performed by any suitable device, in an exemplary embodiment, the steps may be performed by monitoring system 10. At step 502, monitoring system 10 may receive a PPG signal as described herein.

At step 504, monitoring system 10 may generate one or more respiration morphology signals from the PPG signal. In some embodiments, a plurality of respiration morphology signals may be generated from the PPG signal, and the plurality of respiration morphology signals may be selected as described below is step 506. In some embodiments, a particular set of respiration morphology signals may be generated from the PPG signal, for example, in some embodiments, a down signal, a delta of second derivative (DSD) signal, and a kurtosis signal may be generated. Although a respiration morphology signal may be generated in any suitable manner, in an exemplary embodiment, a respiration morphology signal may be generated based on calculating a series of morphology metrics based on a PPG signal. One or more morphology metrics may be calculated for each portion of the PPG signal (e.g., for each fiducial defined portion), a series of morphology metrics may be calculated over time, and the series of morphology metrics may be processed to generate one or more morphology metric signals.

FIG. 6 depicts exemplary signals used for calculating morphology metrics from a received PPG signal. The abscissa of each plot of FIG. 6 may represent time and the ordinate of each plot may represent magnitude. PPG signal 600 may be a received PPG signal, first derivative signal 620 may be a signal representing the first derivative of the PPG signal 600, and second derivative signal 640 may be a signal representing the second derivative of the PPG signal 600. As will be described herein, morphology metrics may be calculated for portions of these signals, and a series of morphology metric calculations calculated over time may be processed to generate the respiration morphology signals. Although particular morphology metric calculations are set forth below, each of the morphology metric calculations may be modified in any suitable manner.

Although morphology metrics may be calculated based on any suitable portions of the PPG signal 600 (as well as the first derivative signal 620, second derivative signal 640, and any other suitable signals that may be generated from the PPG signal 600), in an exemplary embodiment, morphology metrics may be calculated for each fiducial-defined portion such as fiducial defined portion 610 of the PPG signal 600. Exemplary fiducial points 602 and 604 are depicted for PPG signal 600, and fiducial lines 606 and 608 demonstrate the location of fiducial points 602 and 604 relative to first derivative signal 620 and second derivative signal 640.

Although it will be understood that fiducial points may be identified in any suitable manner, in exemplary embodiments fiducial points may be identified based on features of the PPG signal 620 or any derivatives thereof (e.g., first derivative signal 620 and second derivative signal 640) such as peaks, troughs, points of maximum slope, dichrotic notch locations, pre-determined offsets, any other suitable features, or any combination thereof. Fiducial points 602 and 604 may define a fiducial-defined portion 610 of PPG signal 600. The fiducial points 602 and 604 may define starting and ending points for determining morphology metrics, and the fiducial-defined portion 610 may define a relevant portion of data for determining morphology metrics. It will be understood that other starting points, ending points, and relative portions of data may be utilized to determine morphology metrics.

An exemplary morphology metric may be a down metric. The down metric is the difference between a first (e.g., fiducial) sample of a fiducial-defined portion (e.g., fiducial defined portion 610) of the PPG signal (e.g., PPG signal 600) and a minimum sample (e.g., minimum sample 612) of the fiducial-defined portion 610 of the PPG signal 600. The down metric may also be calculated based on other points of a fiducial-defined portion. The down metric is indicative of physiological characteristics which are related to respiration, e.g., amplitude and baseline modulations of the PPG signal. In an exemplary embodiment, fiducial point 602 defines the first location for calculation of a down metric for fiducial-defined portion 610. In the exemplary embodiment, the minimum sample of fiducial-defined portion 610 is minimum point 612, and is indicated by horizontal line 614. The down metric may be calculated by subtracting the value of minimum point 612 from the value of fiducial point 602, and is depicted as down metric 616.

Another exemplary morphology metric may be a kurtosis metric for a fiducial-defined portion. Kurtosis measures the peakedness of the PPG signal 600 or a derivative thereof (e.g., first derivative signal 620 or second derivative signal 640). In an exemplary embodiment, the kurtosis metric may be based on the peakedness of the first derivative signal 620. The peakedness is sensitive to both amplitude and period (frequency) changes, and may be utilized as an input to generate respiration morphology signals that may be used to determine respiration information such as respiration rate. Kurtosis may be calculated based on the following formulae:

$$D = \frac{1}{n} {\sum }_{i = 1}^{n} {( {x}_{i}^{′} - \overline{{x}^{′}} )}^{2}$$

$$Kurtosis = \frac{1}{{nD}^{2}} {\sum }_{i = 1}^{n} {( {x}_{i}^{′} - \overline{{x}^{′}} )}^{4}$$

where:
x₁′=ith sample of 1^{st} derivative;
x̅′̅=mean of 1st derivative of fiducial-defined portion;
n=set of all samples in the fiducial-defined portion

Another exemplary morphology metric may be a delta of the second derivative (DSD) between consecutive fiducial-defined portions, e.g., at consecutive fiducial points. Measurement points 642 and 644 for a DSD calculation are depicted at fiducial points 602 and 604 as indicated by fiducial lines 606 and 608. The second derivative signal is indicative of the curvature of a signal. Changes in the curvature of the PPG signal 600 that can be identified with second derivative signal 640 are indicative of changes in internal pressure that occur during respiration, particularly changes near the peak of a pulse. By providing a metric of changes in curvature of the PPG signal, the DSD morphology metric may be utilized as an input to determine respiration information, such as respiration rate. The DSD metric may be calculated for each fiducial-defined portion by identifying the value of the second derivative signal 640 at the current fiducial point (e.g., fiducial point 642 of fiducial-defined portion 610) and subtracting from that the value of the second derivative signal 640 at the next fiducial point (e.g., fiducial point 644 of fiducial-defined portion 610).

Although a down metric, kurtosis metric, and DSD metric have been described, any suitable morphology metrics related to respiration may be calculated for use in generating respiration morphology signals. Other exemplary morphology metrics that may be relevant to determining a physiological parameter such as respiration information from a PPG signal may include an up metric, a skew metric, a ratio of samples metric (e.g., a b/a ratio metric or c/a ratio metric), a i_b metric, a peak amplitude metric, a center of gravity metric, and an area metric. It will be understood that metrics may be determined from the original PPG signal or any derivative thereof (e.g., a down metric may be determined for each of the PPG signal, the first derivative of the PPG signal, and/or the second derivative of the PPG signal).

In some embodiments, each series of morphology metric values may be further processed in any suitable manner to generate the respiration morphology signals. Although any suitable processing operations may be performed for each series of morphology metric values, in an exemplary embodiment, each series of morphology metric values may be filtered (e.g., based on frequencies associated with respiration) and interpolated to generate the plurality of respiration morphology signals. Processing may then continue to step 506.

At step 506, monitoring system 10 may select respiration morphology signals from the respiration morphology signals generated in step 504. In some embodiments, step 506 may not be performed. For example, a predetermined plurality of respiration morphology signals such as the down morphology signal (for the PPG signal), DSD morphology signal, and kurtosis morphology signal may be generated from the PPG signal and processed to generate one or more principal components and calculate respiration information as described herein. However, in some embodiments, a subset of the plurality of morphology signals generated in step 504 may be selected for further processing in step 506, while other respiration morphology signals may be discarded. If step 506 is performed, the respiration morphology signals from step 504 may be referred to as candidate respiration morphology signals. Although some embodiments for selecting and discarding from the candidate respiration morphology signals are described herein, it will be understood that the embodiments may be combined in any suitable manner, that other suitable methods for selecting or discarding candidate respiration morphology signals may be used in accordance with the present disclosure, and that the other methods may also be combined with the methods described herein in any suitable manner.

Although candidate respiration morphology signals may be selected in any suitable manner, in some embodiments, candidate respiration morphology signals may be selected based on predicted respiration information such as respiration rate. In some embodiments, recent respiration rate values may be analyzed to determine an expected respiration rate range associated with the respiration morphology signals. Although the expected respiration rate range may be determined in any suitable manner, in some embodiments, the expected respiration rate may be based on the trend of a subset of recently determined respiration rate values. Once an expected respiration rate range is determined, a plurality of respiration morphology signals may be selected based on the expected respiration rate. For example, some candidate respiration morphology signals may carry more low rate information such as baseline or frequency modulation, and may be more suitable for determining respiration information when the respiration rate is relatively low, while other candidate respiration morphology signals may be more suitable for determining respiration information when the respiration rate is relatively high. In some embodiments, a down morphology signal may be used for the determination of respiration information at low respiration rates, while a DSD morphology signal or baRatio morphology signal may be used for determination of respiration information at higher rates.

In some embodiments, respiration morphology signals may be selected based on confidence metrics associated with the candidate respiration morphology signals. A confidence metric may be based on any suitable information relevant to identifying a signal as including respiration information, such as signal shape, an expected range of respiration rate, a comparison of a frequency associated with each respiration morphology signals with recently calculated respiration rates, the periodicity of each respiration morphology signal, any other suitable parameter, or any combination thereof. In some embodiments, a plurality of the candidate respiration morphology signals most likely to accurately represent respiration information may be selected based on the confidence metrics.

At step 508, monitoring system 10 may generate one or more principal components from the plurality of respiration morphology signals using principal component analysis (“PCA”). It will be understood that PCA may be performed on any suitable subsets of the respiration morphology signals to generate any suitable number of principal components. For example, in an exemplary embodiment, three principal components may be generated from twelve respiration morphology signals.

In PCA, a plurality of signals that are believed to be correlated are transformed to identify a set of linearly uncorrelated principal components. In the context of the present disclosure, the plurality of signals are the respiration morphology signals. Because each of the respiration morphology is believed to represent respiration information, the respiration morphology signals should be correlated based on the underlying modulation of the PPG signal due to respiration.

PCA may be performed by identifying a variable (i.e., principal component) that represents the maximum variance for the underlying data (e.g., the respiration morphology signals) to identify the first principal component. The second principal component is the variable that has the highest variance while being orthogonal to the first principal component, and so on for additional principal components. In this manner, the principal components that are generated with PCA may be said to be orthogonal signals having ordered maximum variance for the plurality of input signals.

An example of performing PCA to identify one or more principal components associated with a plurality of respiration morphology signals is depicted in FIGS. 7-9. FIG. 7 shows illustrative respiration morphology signals in accordance with some embodiments of the present disclosure, FIG. 8 shows an illustrative attractor in phase space of the three respiration morphology signals from FIG. 7 and two exemplary principal components identified from those signals, and FIG. 9 is an illustrative plot of principal component 1 from FIG. 8.

The abscissa in FIG. 7 may represent time and the ordinate of FIG. 7 may represent magnitude. Each of the respiration morphology signals depicted in FIG. 7 may be generated as described herein. In an exemplary embodiment, the respiration morphology signals may include a down signal 702, a kurtosis signal 704, and a DSD signal 706. The three respiration morphology signals from FIG. 7 may be plotted against each other to form an attractor in phase space as illustrated in FIG. 8.

FIG. 8 shows an illustrative attractor generated from respiration morphology signals in accordance with some embodiments of the present disclosure. Each of the axes of FIG. 8 may represent one of respiration morphology signals (i.e., the axis labeled “Signal 1” may correspond to down signal 702, the axis labeled “Signal 2” may correspond to kurtosis signal 704, and the axis labeled “Signal 3” may correspond to DSD signal 706).

As can be seen in FIG. 8, “PC1” corresponds to the maximum variance for the plotted data and is the first principal component, while “PC2” has the maximum variance in a direction that is orthogonal to PC1 and is the second principal component. Although not depicted, additional principal components could also be determined (i.e., a third principal component would be in the direction having maximum variance and orthogonal to both PC1 and PC2). In some embodiments, the first principal component (PC1) representing the maximum variance for the plurality of respiration morphology signals may correspond to the underlying respiration information signal that created respiratory modulations in the PPG signal.

FIG. 9 shows an illustrative principal component in accordance with some embodiments of the present disclosure. The abscissa in FIG. 9 may represent time and the ordinate of FIG. 9 may represent magnitude. The signal labeled PC1 may correspond to principal component 1. Once the principal component corresponding to respiration information is identified (or if PCA is performed on multiple subsets of respiration modulation signals, once the principal component associated with each subset of respiration morphology signals is identified), processing may continue to step 510 of FIG. 5.

At step 510 monitoring system 10 may calculate respiration information. The respiration information may be calculated based on the principal component, a plurality of principal components, the plurality of respiration morphology signals, or any combination thereof. In some embodiments, respiration information such as respiration rate may be calculated based on the principal component that is identified as corresponding to the respiration source signal (e.g., if a single principal component is determined for a single set of respiration morphology signals as depicted in FIGS. 7-9). Although respiration information such as respiration rate may be calculated from the selected principal component in any suitable manner, in some embodiments, the respiration rate may be calculated based on autocorrelation methods, Fourier analysis, wavelet transforms, any other suitable method, or any combination thereof. For example, in an embodiment, an autocorrelation may be performed for on an identified principal component to generate an autocorrelation sequence. An autocorrelation signal may be generated for each autocorrelation sequence in any suitable manner, such as by interpolating the autocorrelation sequence. Peaks of the autocorrelation signal may correspond to periodicity in the underlying respiration source signal, and the time between selected peaks may represent a period associated with the respiration rate.

In some embodiments, respiration information such as respiration rate may be calculated for one or more principal components and one or more of the respiration morphology signals. In an exemplary embodiment, a confidence value may be calculated for each of the principal components and each of the respiration morphology signals and those signals may be combined. Although signals may be combined in any suitable manner, a combined signal may be generated based on weighting each of the signals based on their relative confidence values. The signals may be combined at any stage of the analysis. For example, in an exemplary embodiment, an autocorrelation signal may be generated for each of the principal components and the one or more respiration morphology signals. The autocorrelation signals may be combined based on the confidence values associated with each one of the signals, and respiration information such as respiration rate may be determined from the combined autocorrelation sequence for example, based on autocorrelation methods, Fourier analysis, wavelet transforms, any other suitable method, or any combination thereof.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A method comprising:
receiving a photoplethysmograph (PPG) signal;
processing, with processing equipment, the PPG signal to generate a plurality of respiration morphology signals;
performing, with the processing equipment, principal component analysis on the plurality of respiration morphology signals to generate one or more principal components;
identifying, with the processing equipment, a principal component of the one or more principal components that corresponds to a respiration source signal; and
determining, with the processing equipment, respiration information based at least in part on the identified principal component.

2. The method of claim 1, wherein the plurality of respiration morphology signals comprise one or more of a down signal, a difference in the second derivative signal, and a kurtosis signal.

3. The method of claim 1, wherein processing the PPG signal to generate a plurality of respiration morphology signals comprises:
generating a plurality of candidate respiration morphology signals;
calculating one or more confidence metrics associated with the candidate respiration morphology signals; and
selecting the plurality of respiration morphology signals from the candidate respiration morphology signals based on the confidence metrics.

4. The method of claim 1, wherein processing the PPG signal to generate a plurality of respiration morphology signals comprises:
generating a plurality of candidate respiration morphology signals;
determining a predicted respiration range; and
selecting the plurality of respiration morphology signals from the candidate respiration morphology signals based on the predicted respiration range.

5. The method of claim 1, wherein the method further comprises:
processing, with processing equipment, the PPG signal to generate a second plurality of respiration morphology signals;
performing, with the processing equipment, principal component analysis on the second plurality of respiration morphology signals to generate one or more second principal components;
identifying, with the processing equipment, a second principal component of the one or more second principal components that corresponds to a respiration source signal; and
determining, with the processing equipment, respiration information based at least in part on the identified principal component and the second identified principal component.

6. The method of claim 1, wherein determining the respiration information comprises:
generating a combined signal based on the identified principal component and one or more of the plurality of respiration morphology signals; and
determining the respiration information based on the combined signal.

7. The method of claim 7, wherein generating the combined signal comprises:
determining a confidence value for each of the identified principal component and the one or more of the plurality of respiration morphology signals; and
generating a combined signal from the identified principal component and the one or more of the plurality of respiration morphology signals based on the confidence values.

8. A non-transitory computer-readable storage medium for use in determining respiration information for a patient, the computer-readable medium having computer program instructions recorded thereon for:
receiving a photoplethysmograph (PPG) signal;
processing the PPG signal to generate a plurality of respiration morphology signals;
performing principal component analysis on the plurality of respiration morphology signals to generate one or more principal components;
identifying a principal component of the one or more principal components that corresponds to a respiration source signal; and
determining respiration information based at least in part on the identified principal component.

9. The computer-readable medium of claim 8, wherein the plurality of respiration morphology signals comprise one or more of a down signal, a difference in the second derivative signal, and a kurtosis signal.

10. The computer-readable medium of claim 8, wherein processing the PPG signal to generate a plurality of respiration morphology signals comprises:
generating a plurality of candidate respiration morphology signals;
calculating one or more confidence metrics associated with the candidate respiration morphology signals; and
selecting the plurality of respiration morphology signals from the candidate respiration morphology signals based on the confidence metrics.

11. The computer-readable medium of claim 8, wherein processing the PPG signal to generate a plurality of respiration morphology signals comprises:
generating a plurality of candidate respiration morphology signals;
determining a predicted respiration range; and
selecting the plurality of respiration morphology signals from the candidate respiration morphology signals based on the predicted respiration range.

12. The computer-readable medium of claim 8, wherein determining the respiration information comprises:
generating a combined signal based on the identified principal component and one or more of the plurality of respiration morphology signals; and
determining the respiration information based on the combined signal.

13. The computer-readable medium of claim 12, wherein generating a combined signal comprises:
determining a confidence value for each of the identified principal component and the one or more of the plurality of respiration morphology signals; and
generating a combined signal from the identified principal component and the one or more of the plurality of respiration morphology signals based on the confidence values.

14. A patient monitoring system comprising processing equipment configured to:
receive a photoplethysmograph (PPG) signal;
process the PPG signal to generate a plurality of respiration morphology signals;
perform principal component analysis on the plurality of respiration morphology signals to generate one or more principal components;
identify a principal component of the one or more principal components that corresponds to a respiration source signal; and
determine respiration information based at least in part on the identified principal component.

15. The patient monitoring system of claim 14, wherein the plurality of respiration morphology signals comprise one or more of a down signal, a difference in the second derivative signal, and a kurtosis signal.

16. The patient monitoring system of claim 14, wherein the patient monitoring system is configured to:
generate a plurality of candidate respiration morphology signals;
calculate one or more confidence metrics associated with the candidate respiration morphology signals; and
select the plurality of respiration morphology signals from the candidate respiration morphology signals based on the confidence metrics.

17. The patient monitoring system of claim 14, wherein the patient monitoring system is configured to:
generate a plurality of candidate respiration morphology signals;
determine a predicted respiration range; and
select the plurality of respiration morphology signals from the candidate respiration morphology signals based on the predicted respiration range.

18. The patient monitoring system of claim 14, wherein the patient monitoring system is configured to:
process the PPG signal to generate a second plurality of respiration morphology signals;
perform principal component analysis on the second plurality of respiration morphology signals to generate one or more second principal components;
identify a second principal component of the one or more second principal components that corresponds to a respiration source signal; and
determine respiration information based at least in part on the identified principal component and the second identified principal component.

19. The patient monitoring system of claim 14, wherein the patient monitoring system is configured to:
generate a combined signal based on the identified principal component and one or more of the plurality of respiration morphology signals; and
determine the respiration information based on the combined signal.

20. The patient monitoring system of claim 14, wherein the patient monitoring system is configured to:
determine a confidence value for each of the identified principal component and the one or more of the plurality of respiration morphology signals;
generate a combined signal from the identified principal component and the one or more of the plurality of respiration morphology signals based on the confidence values; and
determine the respiration information based on the combined signal.
